(19) United States
(12) Patent Application Publication
(10) Pub. No.: US 2004/0180855 A1
(43) Pub. Date: Sep. 16, 2004

(54) Methods of treating thrombosis with reduced risk of increased bleeding times

(76) Inventors: William A. Schumacher, Newtown, PA (US); Steven M. Seiler, Pennington, NJ (US); Jing S. Belfield, Lawrenceville, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/780,819

(22) Filed: Feb. 18, 2004

Related U.S. Application Data
(60) Provisional application No. 60/448,646, filed on Feb. 19, 2003.

(51) Int. Cl. A61K 31/727; A61K 31/40

(52) U.S. Cl. 514/56; 514/423

(57) ABSTRACT

The present invention relates to methods of treating thrombosis in mammals comprising administration of a sufficient amount of a small molecule Factor XIa inhibitor to inhibit thrombosis in the mammal with little or no effect on bleeding times. The invention also relates to pharmaceutical compositions useful in practicing the claimed methods.

### CROSS-REFERENCE TO RELATED APPLICATIONS

The present application claims the priority benefit of U.S. Provisional Application No. 60/448,646, filed Feb. 19, 2003, which is expressly incorporated fully herein by reference.

### FIELD OF THE INVENTION

The present invention relates to methods of treating thrombosis in mammals comprising administration of a sufficient amount of a small molecule Factor XIa inhibitor to inhibit thrombosis in the mammal with little or no effect on bleeding times. The invention also relates to pharmaceutical compositions useful in practicing the claimed methods.

### BACKGROUND OF THE INVENTION

Thrombo-embolic disorders are the largest cause of mortality (myocardial infarction) and disability (stroke) in the industrialized world. Arterial thrombosis is initiated by atherosclerotic plaque rupture, exposure of tissue factor, and initiation of the coagulation vortex. A number of coagulation factors are present in the blood as precursors (e.g., Factors VII-XII), and when the coagulation system is triggered, these factors undergo a complicated, ordered series of reactions that ultimately lead to thrombin production. Thrombin is a proteolytic enzyme that occupies a central position in the coagulation process. Thrombin catalyzes the conversion of fibrinogen to fibrin, is a key effector enzyme for blood clotting, and is also pivotal for other functions. High concentrations of thrombin inhibit fibrinolysis by activating the Thrombin Activated Fibrinolysis Inhibitor (TAFI), which can also be activated by modest amounts of thrombin in the presence of soluble or membrane bound thrombomodulin. TAFIa removes the C-terminal lysine residues from fibrin, preventing the binding of t-PA and plasmin and thus, slowing fibrinolysis.

The complicated coagulation process is initiated by tissue factor (TF). Tissue factor binds and activates Factor VII (FVII), which is rapidly converted to activated Factor VIIa (FVIIa) to form a TF:FVIIa complex. The TF:FVIIa complex activates Factors IX and X. Factor Xa generates small amounts of thrombin. The small amounts of thrombin activate Factor V, Factor VIII and platelets, accelerating thrombin production by Factors IXa and Xa. Activation of Factor V and FVIII accelerates catalytic activity of FVIIIa:FIXa and FVa:FXa, resulting in dramatically increased thrombin production. Another wave of thrombin generation occurs as a result of thrombin activation of Factor XIa. Factor XI activates more Factor IX. As the concentration of thrombin increases, more thrombin is generated, which in turn activates TAFI to then inhibit fibrinolysis.

This coagulation process involves an intrinsic pathway and an extrinsic pathway. In the intrinsic pathway, Factor XII (aka Hageman Factor) is converted from its inactive form (zymogen) to an active form, i.e., Factor XIIa. Activated Factor XII enzymatically activates Factor XI to Factor XIa. Activated Factor XI activates Factor IXa. Factor IXa then converts Factor X to Factor Xa. FXa activates prothombin to thrombin. Thrombin cleaves fibrinogen to form insoluble fibrin (the clot). In the extrinsic pathway, addition of thromboplastin (i.e., tissue factor) to plasma activates Factor VII. This complex, in the presence of calcium ions and phospholipids, activates Factor X to Factor Xa. Once Factor Xa is generated, the remainder of the cascade is similar to the intrinsic pathway. As can be seen, Factor XIa is involved only in the intrinsic pathway.

In vitro, the degree to which FXIa contributes to thrombin generation, platelet activation, and fibrin formation depends on the concentration of tissue factor. For example, in the absence of FXI (i.e., in FXIa deficient plasma), plasma stimulated with low levels of tissue factor (clot formation >10 minutes) showed a delay in the time required to generate thrombin and form clots. A FXI deficiency also decreased the amount of thrombin generated and platelet aggregation in whole blood. However, in blood or plasma stimulated by higher concentration of tissue factor (clot formation <5 minutes), a FXI deficiency had no effect on the thrombin generation or clot formation. Thus, a FXI deficiency will generally prolong thrombin generation but not in situations where the plasma is stimulated with high concentrations of tissue factor.

FXIa, via expanded thrombin generation, also plays a role in resisting fibrinolysis. Resistance of plasma clots to tPA and uPA-induced fibrinolysis depends on thrombin concentration (generated endogenously or added exogenously) in the plasma. The time required for clot lysis is proportional to the plasma TAFIa concentrations. However, clot lysis can occur more rapidly, and the lysis made independent of plasma TAFI concentration, when blocking antibodies to FXIa are included in the assay.

Elevated levels of FXIa in the plasma and/or increased activation of FXIa is associated with various cardiovascular and other diseases. As an illustration, increased activation of FXIa occurs in patients with coronary artery disease and is related to the severity of the disease. Also, Factor IX activation peptide (a product of FXIa and TF:FVIIa cleavage of FIX) levels have been found to be significantly higher in patients with acute myocardial infarction and unstable angina compared with patients with stable angina. Concentrations of FXIa-α₁AT (FXIa complexed to the serpin α1-antitrypsin) were also elevated in patients with recent myocardial infarction or unstable angina. Patients with high levels of Factor XI are at risk factor for deep venous thrombosis.

Proteins or peptides that reportedly inhibit Factor XIa are disclosed in WO 01/27079 to Entremed, Inc. There are advantages in using small organic compounds, however, in preparing pharmaceuticals, e.g., small compounds generally have better oral bioavailability and compatibility in making formulations to aid in delivery of the drug as compared with large proteins or peptides. Small organic compounds have been disclosed that reportedly inhibit coagulation factors besides Factor XIa. For example, compounds effective in inhibiting Factor Xa are described in U.S. Pat. Nos. 6,344,450 and 6,297,233, and WO 00/47563. Compounds effective in inhibiting Factors VIIa, Xa, as well as tryptase and urokinase are described in U.S. Pat. No. 6,335,324. Factor Xa inhibitors are disclosed in WO 98/57937 to the duPont Merck Pharmaceutical Co., and Factor VIIa inhibitors are disclosed in U.S. Pat. No. 6,358,960 to Ono Pharmaceuticals Inc., (“Ono”), and in WO 01/44172 to Axys Pharm. Inc. Small molecular inhibitors of Factor XIa are described in WO 02/42273 to the present assignee.

A possible adverse side effect associated with use of anti-thrombotic agents for treating cardiovascular diseases involves the risk of bleeding. For example, heparin is a known anti-thrombotic agent that has a highly-variable dose-related response, and its anticoagulant effects must be closely monitored to avoid a risk of serious bleeding. The erratic anticoagulant response of heparin is likely due to its propensity to bind non-specifically to plasma proteins. Aspirin also has been used as an anti-thrombotic agent but at high doses presents a risk of gastrointestinal bleeding. Thrombin inhibitors and their drawbacks are further discussed in WO 96/20689 to duPont Merck Pharmaceutical Co. Guanidine and beta lactam-containing compounds that are potent inhibitors of serine proteases including thrombin and tryptase are described in U.S. Pat. No. 6,335,324, the entire contents of which is incorporated herein by reference.

### SUMMARY OF THE INVENTION

The inventors have surprisingly discovered that selective inhibition of clotting Factor XIa with a small organic compound according to the invention inhibits both venous and arterial thrombosis with no or substantially no effect on small vessel bleeding time and/or little or no impairment of haemostasis. According to one aspect of the invention, there is provided a method of treating thrombosis in a mammal by administering to the mammal a small organic compound effective for inhibiting coagulation Factor XIa. Advantageously, the method comprises administering to the mammal a small organic compound having activity for inhibiting Factor XIa with an IC₅₀ value of below 120 nM, more preferably below 10 nM, and even more preferably below 1 nM.

According to another aspect of the invention, there is provided a method of treating thrombosis in a mammal comprising administering to the mammal of a pharmaceutical composition that inhibits thrombosis in the mammal, wherein the pharmaceutical composition contains a sufficient amount of a small compound that is selective for inhibition of Factor XIa inhibitor so that the differential rate of percent inhibition of thrombosis in the mammal is greater than the differential rate of percent increase in bleeding time. Advantageously, the differential rate of thrombosis inhibition is at least 25 percent greater than the differential rate of percent increase in bleeding time, more preferably at least 50 percent greater.

### BRIEF DESCRIPTION OF THE DRAWINGS

The following drawings are set forth herein to better illustrate the invention without limitating the scope thereof:

FIG. 1 is a graph showing the carotid blood flow (ml/min) as a function of time upon i.v. administration of saline (control) and a selective Factor XIa inhibitor, following the arterial thrombosis model described herein;

FIG. 2 is a bar graph showing the reduction in thrombus weight (mg) for carotid arteries of rats subjected to the arterial thrombosis model described for FIG. 1);

FIG. 3 is a bar graph showing the mass of thrombus (measured gravimetrically) formed upon administration of a Factor XIa inhibitor in a venous thrombosis (FeCL₂-VT) model in rats;

FIG. 4 is a bar graph showing the mass of thrombus formed upon administration of a Factor XIa inhibitor in a venous thrombosis (stasis-VT) model in rats;

FIGS. 5 and 6 are bar graphs showing pre- and post-treatment bleeding times for rats in renal cortex and mesenteric artery bleeding time models, respectively, upon administration of a Factor XIa inhibitor; and

FIG. 7 is a graph showing the differential effect on thrombosis and prolongation of bleeding time in anesthetized rats upon administration of thrombin and FXa inhibitors as compared with a FXIa inhibitor.

### DETAILED DESCRIPTION OF THE INVENTION

The following are definitions of terms used in this specification. The initial definition provided for a group or term herein applies to that group or term throughout the present specification, individually or as part of another group, unless otherwise indicated.

The term “APTT” as used herein means “activated partial thromboplastin times” which is intended to refer to the time elapsed between the formation of an assay solution and the detection of clot formation in the assay solution with use of an APTT assay, i.e., an assay employing an intrinsic pathway activator, such as micronized silica, and a phospholipid component of a thromboplastin reagent (without tissue factor protein) for evaluating coagulation associated with the intrinsic pathway.

The term “PT” time as used herein refers to prothrombin times which is intended to refer to the time elapsed between formation of an assay solution and detection of clot formation in the assay solution with use of a PT assay, i.e., an assay employing thromboplastin reagents that are used for evaluating blood coagulation associated with the extrinsic pathway.

The term “bleeding time” as used herein means the elapsed time between an injury involving severance of a small blood vessel and the cessation of bleeding. A small blood vessel would present in tissue (arteriole, capillary, venule) as distinguished from a large blood that carries blood to an organ or tissue perfusion bed (e.g. coronary or carotid artery, femoral or brachial vein). When it is stated herein that there is “no substantial effect” on bleeding time, it is meant that when a given dose of a pharmaceutical agent is administered to a subject, on average there is less than a 30% change in pre-treatment and post-treatment bleeding times when measured by vessel severance as is typically accomplished with a template incision device. A 30% to 50% increase in bleeding time is typically what we observe in the rat experimental models in response to aspirin and what is commonly seen in human subject with forearm template incisions. This is the smallest increase in bleeding time that we can statistically detect experimentally.

The term “haemostasis” as used herein means the rate at which small blood vessels are maintained intact in a mammal under normal conditions, i.e., under conditions that do not involve administration of an anticoagulant or anti-thrombotic agent. The ability of a mammal to maintain haemostatis means the ability of the mammal to prevent or avoid blood loss caused by severance of small blood vessels. When it is stated herein that there is little impairment of haemostasis, it is meant that upon administration of a given dose of a pharmaceutical agent to a subject, the subject's ability to prevent blood loss caused by severence of a small blood vessel post-treatment is impaired by less than 30 percent as compared with pre-treatment, i.e., there is a less than a statistically detectable 30 percent increase in bleeding times as defined above with respect to small blood vessels.

The term “potent” as used herein with reference to a compound that inhibits Factor XIa means the compound exhibited an efficacy for inhibiting Factor XIa in vitro with an IC₅₀ of less than 120 nM. The term “highly potent” or “high potenncy” as used herein means the compound exhibited an efficacy for inhibiting Factor XIa in vitro with an IC₅₀ of less than 10 nM.

The term “large blood vessel” as used herein means a blood vessel that carries blood to an organ or tissue perfusion bed (e.g. coronary or carotid artery, femoral or brachial vein). As compared with a “small blood vessel” which is intended to refer to a blood vessel present in tissue (arteriole, capillary, venule).

The terms “small compound” or “small molecule” as used herein means a non-peptidic organic compound having less than 1000 molecular weight, with preferred compounds having less than 750 molecular weight, and even more preferred compounds having less than 500 molecular weight.

The term “selective” as used herein with respect to inhibition of Factor XIa means the compound has a ten-fold greater effect in inhibiting Factor XIa in plasma as compared with TF:FVIIa, FXIIa, FIXa, FXa, thrombin, plasmin, urokinase, and tissue plasminogen activator. “Highly selective” or “high selectivity” as used herein with respect to inhibition of Factor XIa means the compound has a 100-fold greater effect in inhibiting Factor XIa as compared with these other blood coagulation factors. When it is stated herein that a compound is comparatively selective for inhibition of Factor XIa, it is meant that the compound has a 1000-fold greater effect in inhibiting Factor XIa as compared with the direct-acting coagulation protease inhibitors compounds 16A and 16B, described hereinafter.

The term “alkyl” refers to straight or branched chain hydrocarbon groups having 1 to 12 carbon atoms, preferably 1 to 8 carbon atoms. Lower alkyl groups, that is, alkyl groups of 1 to 4 carbon atoms, are most preferred.

The term “substituted alkyl” refers to an alkyl group as defined above having one, two, or three substituents selected from the group consisting of halogen, trifluoromethyl, trifluoromethoxy, alkenyl, alkynyl, nitro, cyano, keto (═O), —ORₐ, —SRₐ, —NRₐR_{b}, —NRₐSO₂R_{c}, —SO₂R_{c}, —SO₂NRₐR_{b}, —CO₂Rₐ, —C(═O)Rₐ, —C(═O)NRₐR_{b}, —OC(═O)Rₐ, —OC(═O)NRₐR_{b}, —NRₐC(═O)R_{b}, —NRₐCO₂R_{b}, cycloalkyl, heterocyclo, aryl, and heteroaryl, wherein Rₐ and R_{b} are selected from hydrogen, alkyl, alkenyl, cycloalkyl, heterocyclo, aryl, and heteroaryl, and R_{c} is selected from alkyl, alkenyl, cycloalkyl, heterocyclo, aryl and heteroaryl. When a substituted alkyl includes a cycloalkyl, heterocyclo, aryl, or heteroaryl substituent, said ringed systems are as defined below and thus may in turn have zero to three substituents (preferably 0-2 substituents), also as defined below. When either Rₐ, R_{b} or R_{c} is an alkyl or alkenyl, said alkyl or alkenyl may optionally be substituted with 1-3 of halogen, trifluoromethyl, trifluoromethoxy, nitro, cyano, keto (═O), OH, alkoxy, phenyloxy, benzyloxy, SH, S(alkyl), NH₂, —NH(alkyl), —N(alkyl)₂, —NHSO₂(alkyl), —SO₂(alkyl), —SO₂NH₂, —SO₂NH(alkyl), —SO₂N(alkyl)₂, —CO₂H, —CO₂(alkyl), —C(═O)H, —C(═O)alkyl, —C(═O)NH₂, —C(═O)NH(alkyl), —C(═O)N(alkyl)₂, —OC(═O)alkyl, —OC(═O)NH₂, —OC(═O)NH(alkyl), —NHC(═O)alkyl, and/or —NHCO₂(alkyl).

“Alkyl” when used in conjunction with another group such as in “arylalkyl” or “cycloalkylalkyl” refers to a substituted alkyl in which at least one of the substituents is the specifically-named group. For example, the term arylalkyl includes benzyl, or any other straight or branched chain substituted alkyl having at least one aryl group attached at any point of the alkyl chain.

The term “alkenyl” refers to straight or branched chain hydrocarbon groups having 2 to 12 carbon atoms and at least one double bond. Alkenyl groups of 2 to 6 carbon atoms and having one double bond are most preferred.

The term “alkylene” refers to bivalent straight or branched chain hydrocarbon groups having 1 to 12 carbon atoms, preferably 1 to 8 carbon atoms, e.g., {—CH₂—}ₙ, wherein n is 1 to 12, preferably 1-8. Lower alkylene groups, that is, alkylene groups of 1 to 4 carbon atoms, are most preferred. The terms “alkenylene” and “alkynylene” refer to bivalent radicals of alkenyl and alknyl groups, respectively, as defined above.

When reference is made to a substituted alkylene, alkenylene, or alkynylene group, these groups are substituted with one to three substitutents as defined above for alkyl groups.

The term “alkoxy” refers to an alkyl or substituted alkyl group bonded through an oxygen atom (—O—). For example, the term “alkoxy” includes the groups —O-C₁₋₁₂alkyl, —O—CH₂aryl, and so forth. A lower alkoxy includes methoxy, ethoxy, propyloxy, and butoxy, wherein the alkyl portion may be straight or branched chain.

The term “alkylthio” refers to an alkyl or substituted alkyl group bonded through a sulfur (—S—) atom. For example, the term “alkylthio” includes the groups —S—(CH₂)CH₃, —S—CH₂aryl, etc.

The term “alkylamino” refers to an alkyl or substituted alkyl group bonded through a nitrogen (—NR′—) group. For example, the term “alkylamino” includes the groups —NR′-C₁₋₁₂ alkyl and —NR′—CH₂-aryl, etc. (where R′ is hydrogen, alkyl or substituted alkyl as defined above.) “Amino” refers to the group —NH₂. The term “aminoalkyl” means a substituted alkyl having at least one amino substituent. “Alkylaminoalkyl” means a substituted alkyl having at least one alkylamino substituent.

When a subscript is used as in C₁₋₈alkyl, the subscript refers to the number of carbon atoms the group may contain. Zero when used in a subscript denotes a bond, e.g., C₀₋₄ alkyl refers to a bond or an alkyl of 1 to 4 carbon atoms. Thus, for example, “C₁₋₆ alkyl” refers to straight and branched chain alkyl groups with one to six carbon atoms, such as methyl, ethyl, n-propyl, isopropyl, n-butyl, t-butyl, n-pentyl, and so forth. “Hydroxy C₀₋₂ alkyl” includes hydroxy, hydroxymethyl, and hydroxyethyl. When used with alkoxy, thioalkyl, or alkylamino, a subscript refers to the number of carbon atoms that the group may contain in addition to heteroatoms. Thus, for example, monovalent C₁₋₂ alkylamino includes the groups —NH(CH₃), —NH(CH₂CH₃), and —N(CH₃)₂. A lower alkylamino comprises an alkylamino having one to four carbon atoms.

The term “halo” or “halogen” refers to chloro, bromo, fluoro and iodo.

The term “haloalkyl” means a substituted alkyl having one or more halo substituents. For example, “haloalkyl” includes mono, bi, and trifluoromethyl.

The term “haloalkoxy” means an alkoxy group having one or more halo substituents. For example, “haloalkoxy” includes OCF₃.

The term “cycloalkyl” refers to fully saturated and partially unsaturated hydrocarbon rings of 3 to 9, preferably 3 to 7 carbon atoms. The term “cycloalkyl” includes such rings having zero to three substituents (preferably 0-2 substituents), selected from 1) R_{g}; and 2) C₁₋₆ alkyl substituted with one to three R_{g}, wherein R_{g} is selected from the group consisting of halogen, alkyl, alkenyl, substituted alkenyl, alkynyl, nitro, cyano, keto (═O), —ORₐ, —SRₐ, —NRₐR_{b}, —NRₐSO₂R_{c}, —SO₂R_{c}, —SO₂NRₐR_{b}, —CO₂Rₐ, —C(═O)Rₐ, —C(═O)NRₐR_{b}, —OC(═O)Rₐ, —OC(═O)NRₐR_{b}, —NRₐC(═O)R_{b}, —NRₐCO₂R_{b}, aryl, heteroaryl, heterocyclo, and/or another 4 to 7 membered cycloalkyl ring, wherein Rₐ, R_{b} and R_{c} are defined as above. When Rₐ, R_{b} and R_{c} are selected from an alkyl or alkenyl group, such groups are in turn optionally substituted as set forth above in the definition for substituted alkyl. The term “cycloalkyl” also includes such rings having a second ring fused thereto (e.g., including benzo, heterocyclo, or heteroaryl rings) or having a carbon-carbon bridge of 3 to 4 carbon atoms. When a cycloalkyl has a second ring fused thereto or is substituted with a further ring, i.e., aryl, arylalkyl, heteroaryl, heteroarylalkyl, heterocyclo, heterocycloalkyl, cycloalkylalkyl, or a further cycloalkyl ring, such ring in turn may be substituted with one to two C₀₋₆ alkyl substituted with one to two of (or bonded to one of) halogen, trifluoromethyl, C₂₋₆ alkenyl, nitro, cyano, keto (═O), OH, O(alkyl), phenyloxy, benzyloxy, SH, S(alkyl), NH₂, —NH(alkyl), —N(alkyl)₂, —NHSO₂(alkyl), —SO₂(alkyl), —SO₂NH₂, —SO₂NH(alkyl), —SO₂N(alkyl)₂, —CO₂H, —CO₂(alkyl), —C(═O)H, —C(═O)alkyl, —C(═O)NH₂, —C(═O)NH(alkyl), —C(═O)N(alkyl)₂, —OC(═O)alkyl, —OC(═O)NH₂, —OC(═O)NH(alkyl), —NHC(═O)alkyl, and —NHCO₂(alkyl).

Thus, the term “cycloalkyl” includes cyclopropyl, cyclobutyl, cyclopentyl, cyclohexyl, cycloheptyl, cyclooctyl, etc., as well as the following ring systems:

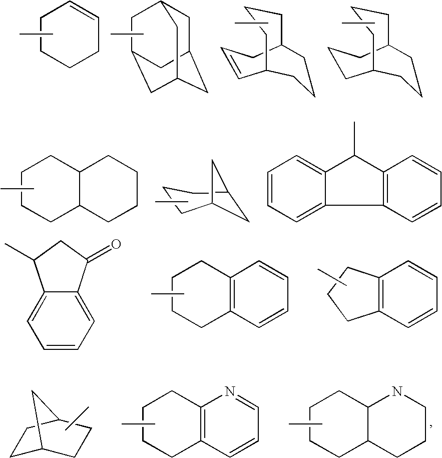

and the like, which optionally may be substituted at any available atoms of the ring(s).

The term “aryl” refers to phenyl, biphenyl, 1-naphthyl, and 2-naphthyl, with phenyl being preferred. The term “aryl” includes such rings having zero to three substituents (preferably 0-2 substituents), selected from the group consisting of 1) Rₕ; and 2) C₁₋₆ alkyl substituted with one to three R_{g}, wherein R_{g} is as defined above for cycloalkyl, and Rₕ is selected from the same groups as R_{g} but does not include keto (═O). Additionally, two substituents attached to an aryl, particularly a phenyl group, may join to form a further ring such as a fused or spiro-ring, e.g., cyclopentyl or cyclohexyl, or fused heterocycle or heteroaryl. When an aryl is substituted with a further ring, such ring in turn may be substituted with one to two C₀₋₆alkyl substituted with one to two of (or bonded to one of) halogen, trifluoromethyl, C₂₋₆alkenyl, nitro, cyano, keto (═O), OH, O(alkyl), phenyloxy, benzyloxy, SH, S(alkyl), NH₂, —NH(alkyl), —N(alkyl)₂, —NHSO₂(alkyl), —SO₂(alkyl), —SO₂NH₂, —SO₂NH(alkyl), —SO₂N(alkyl)₂, —CO₂H, —CO₂(alkyl), —C(═O)H, —C(═O)alkyl, —C(═O)NH₂, —C(═O)NH(alkyl), —C(═O)N(alkyl)₂, —OC(═O)alkyl, —OC(═O)NH₂, —OC(═O)NH(alkyl), —NHC(═O)alkyl, and —NHCO₂(alkyl).

Thus, examples of aryl groups include:

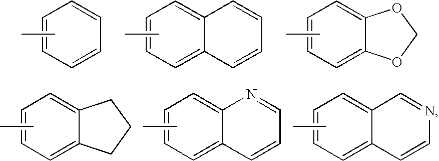

and the like, which optionally may be substituted at any available carbon atom.

The term “heterocyclo” refers to substituted and unsubstituted non-aromatic 3 to 7 membered monocyclic groups, 7 to 11 membered bicyclic groups, and 10 to 15 membered tricyclic groups, in which at least one of the rings has at least one heteroatom (O, S or N). Each ring of the heterocyclo group containing a heteroatom can contain one or two oxygen or sulfur atoms and/or from one to four nitrogen atoms provided that the total number of heteroatoms in each ring is four or less, and further provided that the ring contains at least one carbon atom. The fused rings completing bicyclic and tricyclic groups may contain only carbon atoms and may be saturated, partially saturated, or unsaturated. The nitrogen and sulfur atoms may optionally be oxidized and the nitrogen atoms may optionally be quatemized. The heterocyclo group may be attached at any available nitrogen or carbon atom. The heterocyclo ring may contain zero to three substituents (preferably 0-2 substituents), selected from 1) R_{g}; and 2) C₁₋₆ alkyl substituted with one to three R_{g}, wherein R_{g} is defined as above for cycloalkyl groups. Additionally, when a heterocyclo is substituted with a further ring, such ring in turn may be substituted with one to two C₀₋₆alkyl substituted with one to two of (or bonded to one of) halogen, trifluoromethyl, C₂₋₆alkenyl, nitro, cyano, keto (═O), OH, O(alkyl), phenyloxy, benzyloxy, SH, S(alkyl), NH₂, —NH(alkyl), —N(alkyl)₂, —NHSO₂(alkyl), —SO₂(alkyl), —SO₂NH₂, —SO₂NH(alkyl), —SO₂N(alkyl)₂, —CO₂H, —CO₂(alkyl), —C(═O)H, —C(═O)alkyl, —C(═O)NH₂, —C(═O)NH(alkyl), —C(═O)N(alkyl)₂, —OC(═O)alkyl, —OC(═O)NH₂, —OC(═O)NH(alkyl), —NHC(═O)alkyl, and —NHCO₂(alkyl).

Thus, exemplary heterocyclic groups include, without limitation:

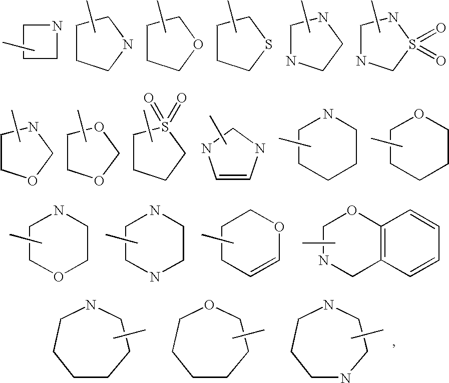

and the like, which optionally may be substituted at any available carbon or nitrogen atom.

The term “heteroaryl” refers to substituted and unsubstituted aromatic 5 to 7 membered monocyclic groups, 9 or 10 membered bicyclic groups, and 11 to 14 membered tricyclic groups which have at least one heteroatom (O, S or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. The heteroaryl ring system may contain zero to three substituents (preferably 0-2 substituents), selected from 1) Rₕ; and 2) C₁₋₆ alkyl substituted with one to three R_{g}, wherein R_{g} and Rₕ are defined above as for aryl groups. Additionally, when a heteroaryl is substituted with a further ring, such ring in turn may be substituted with one to two C₀₋₆alkyl substituted with one to two of (or bonded to one of) halogen, trifluoromethyl, C₂₋₆alkenyl, nitro, cyano, keto (═O), OH, O(alkyl), phenyloxy, benzyloxy, SH, S(alkyl), NH₂, NH(alkyl), N(alkyl)₂, NHSO₂(alkyl), SO₂(alkyl), SO₂NH₂, SO₂NH(alkyl), SO₂N(alkyl)₂, CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and NHCO₂(alkyl).

Examples of heteroaryl rings include

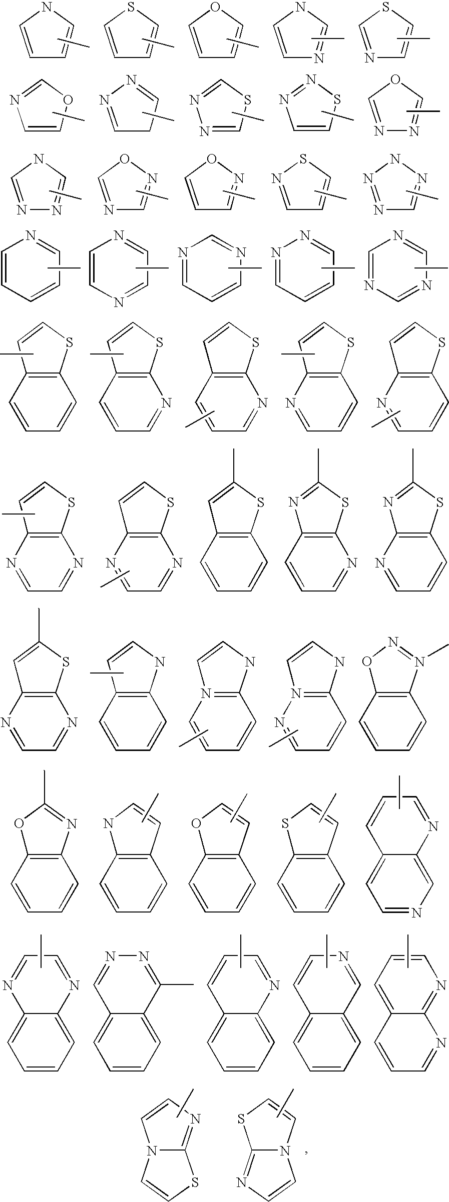

the like, which optionally may be substituted at any available carbon or nitrogen atom.

The term “carbocyclic” refers to optionally substituted aromatic or non-aromatic 3 to 7 membered monocyclic and 7 to 11 membered bicyclic groups, in which all atoms of the ring or rings are carbon atoms.

When the term “unsaturated” is used herein to refer to a ring or group, the ring or group may be fully unsaturated or partially unsaturated.

It should be understood that one skilled in the field may make various substitutions for groups recited in the claims herein, without departing from the spirit or scope of the invention.

Throughout the specification, groups and substituents thereof may be chosen by one skilled in the field to provide stable moieties and compounds.

The compounds of Formula (I) form salts which also may be administered within the scope of this invention. Unless otherwise indicated, reference to a compound for administration according to the invention is understood to include reference to salts thereof. The term “salt(s)” denotes acidic and/or basic salts formed with inorganic and/or organic acids and bases. In addition, the term “salt(s) may include zwitterions (inner salts), e.g., when a compound of formula (I) contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps which may be employed during preparation, and thus, are contemplated within the scope of the invention. Salts of the compounds of the formula (I) may be formed, for example, by reacting a compound of the formula (I) with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts; alkaline earth metal salts such as calcium and magnesium salts; barium, zinc, and aluminum salts; salts with organic bases (for example, organic amines) such as trialkylamines such as triethylamine, procaine, dibenzylamine, N-benzyl-β-phenethylamine, 1-ephenamine, N,N′-dibenzylethylene-diamine, dehydroabietylamine, N-ethylpiperidine, benzylamine, dicyclohexylamine or similar pharmaceutically acceptable amines and salts with amino acids such as arginine, lysine and the like. Basic nitrogen-containing groups may be quatemized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Preferred salts include monohydrochloride, hydrogensulfate, methanesulfonate, phosphate or nitrate.

It is also contemplated that prodrugs and solvates of the compounds specifically identified herein may be administered according to the invention. The term “prodrug” denotes a compound which, upon administration to a subject, undergoes chemical conversion by metabolic or chemical processes to yield a compound of the formula (I), and/or a salt and/or solvate thereof. Various forms of prodrugs are well known in the art. For examples of such prodrug derivatives, see:

a) Design of Prodrugs, edited by H. Bundgaard, (Elsevier, 1985) and Methods in Enzymology, Vol. 42, p. 309-396, edited by K. Widder, et al. (Acamedic Press, 1985);

b) A Textbook of Drug Design and Development, edited by Krosgaard-Larsen and H. Bundgaard, Chapter 5, “Design and Application of Prodrugs,” by H. Bundgaard, p. 113-191 (1991); and

c) H. Bundgaard, Advanced Drug Delivery Reviews, 8, 1-38 (1992), each of which is incorporated herein by reference.

Compounds containing a carboxy group can form physiologically hydrolyzable esters which serve as prodrugs by being hydrolyzed in the body to yield formula (I) compounds per se. Such prodrugs are preferably administered orally since hydrolysis in many instances occurs principally under the influence of the digestive enzymes. Parenteral administration may be used where the ester per se is active, or in those instances where hydrolysis occurs in the blood. Examples of physiologically hydrolyzable esters of compounds of formula (I) include C₁₋₆ alkylbenzyl, 4-methoxybenzyl, indanyl, phthalyl, methoxymethyl, C₁₋₆ alkanoyloxy-C₁₋₆ alkyl, e.g. acetoxymethyl, pivaloyloxymethyl or propionyloxymethyl, C₁₋₆ alkoxycarbonyloxy-C₁₋₆alkyl, e.g methoxycarbonyl-oxymethyl or ethoxycarbonyloxymethyl, glycyloxymethyl, phenylglycyloxymethyl, (5-methyl-2-oxo-1,3-dioxolen-4-yl)-methyl and other well known physiologically hydrolyzable esters used, for example, in the penicillin and cephalosporin arts. Such esters may be prepared by conventional techniques known in the art.

Compounds of formula (I) and salts thereof may exist in their tautomeric form, in which hydrogen atoms are transposed to other parts of the molecules and the chemical bonds between the atoms of the molecules are consequently rearranged. It should be understood that the all tautomeric forms, insofar as they may exist, are included within the invention. Additionally, compounds may have trans and cis isomers and may contain one or more chiral centers, therefore existing in enantiomeric and diastereomeric forms. The invention includes administration of all such isomers, as well as mixtures of cis and trans isomers, mixtures of diastereomers and racemic mixtures of enantiomers (optical isomers). When no specific mention is made of the configuration (cis, trans or R or S) of a compound (or of an asymmetric carbon), then any one of the isomers or a mixture of more than one isomer is intended. The processes for preparation can use racemates, enantiomers or diastereomers as starting materials. When enantiomeric or diastereomeric products are prepared, they can be separated by conventional methods for example, chromatographic or fractional crystallization.

The compounds of the instant invention may, for example, be in the free or hydrate form, and may be obtained by methods exemplified by the following descriptions.

### PREFERRED METHODS AND COMPOSITIONS

FXIa inhibition according to the invention represents a more effective and safer method of inhibiting thrombosis compared to inhibiting other coagulation serine proteases such as thrombin or Factor Xa. According to the discoveries described herein, administration of a small molecule FXIa inhibitor has the effect of inhibiting thrombin generation and clot formation with no or substantially no effect on bleeding times and little or no impairment of haemostasis. These results differ substantially from that of other “direct acting” coagulation protease inhibitors (active-site inhibitors of thrombin and Factor Xa), which demonstrate prolongation of bleeding time and less separation between antithrombotic efficacy and bleeding time prolongation.

A preferred method according to the invention comprises administering to a mammal a pharmaceutical composition containing at least one small molecule inhibitor of Factor XIa having the formula (I),

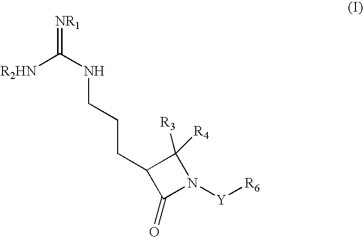

wherein:

R₁ and R₂ are hydrogen;

R₃ is hydrogen or CH₃;

R₄ is selected from hydrogen, CH₃, —CO₂R₇, —C(═O)NR₈R₉, phenyl, benzyl, and phenylethyl, wherein R₇ is hydrogen, C₁₋₆alkyl, benzyl, or —CH(OCOCH₃)CH₃; and each R₄ group is optionally substituted with one to two R₁₂;

Y is C(═O) or —SO₂—; wherein when Y is C(═O), then R₆ is C₁₋₆alkyl, aryl, heteroaryl, or —NR₁₀R₁₁, and when Y is —SO₂—, then R₆ is aryl or heteroaryl; and each R₆ group is optionally substituted with one to two R₁₂;

R₈ and R₉ are individually selected from hydrogen and C₁₋₆alkyl, or R₈ and R₉ taken together form a five or six membered heterocyclo ring optionally substituted with one to two R₁₂ and up to one R₁₃;

R₁₀ and R₁₁ are individually selected from hydrogen, phenyl, or C₁₋₆alkyl optionally substituted with phenyl, or R₁₀ and R₁₁ taken together form a five or six membered heterocyclo ring optionally substituted with one to two R₁₂ and up to one R₁₃;

R₁₂ is selected from hydrogen, halogen, trifluoromethyl, trifluoromethoxy, lower alkyl, amino, lower alkylamino, —CO₂H, —CO₂(lower alkyl), or a five or six membered saturated or unsaturated heterocyclo having up to two nitrogen heteroatoms;

R₁₃ is selected from —C(═O)(C₁₋₆alkyl), —CO₂(C₁₋₆alkyl), —C(═O)NH(C₁₋₆alkyl), and five or six membered heterocyclo optionally substituted with one to two two R₁₄; and

R₁₄ is selected from hydrogen, phenyl, or C₁₋₆alkyl optionally substituted with phenyl;

or a progdrug carbamate thereof wherein at least one of R₁ and R₂ is —COOR, wherein R is hydrogen, C₁₋₆alkyl, benzyl, or —CH(OCOCH₃)CH₃, or a pharmaceutically-acceptable salt or hydrate of said compound or prodrug carbamate.

Further preferred methods comprise administering to a mammal a pharmaceutical composition containing a compound having the formula (Ia),

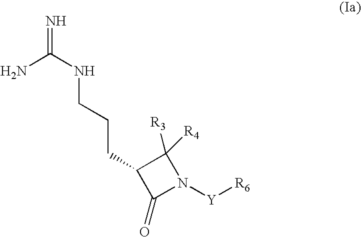

wherein:

R₃ is hydrogen or CH₃;

R₄ is CH₃, —CO₂H, —CO₂(C₁₋₄alkyl),

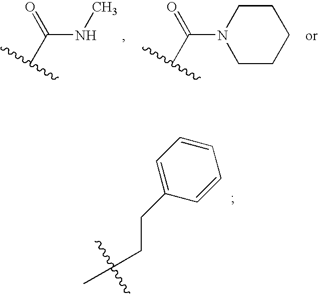

Y is C(═O) or —SO₂—; wherein:

when Y is C(═O), then R₆ is methyl, ethyl propyl,

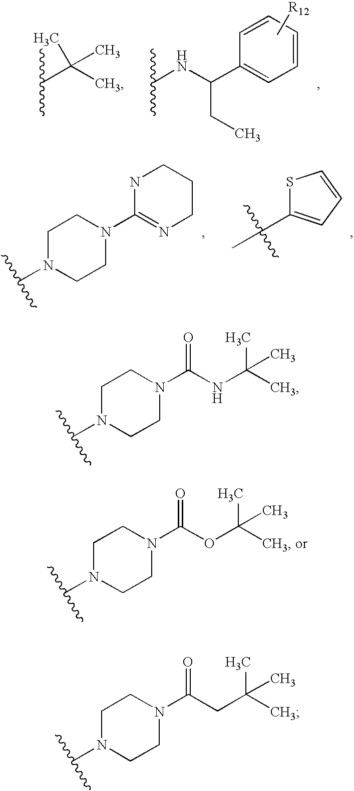

when Y is —SO₂—, then R₆ is

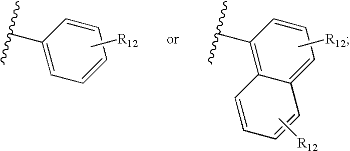

and

R₁₂ is selected from hydrogen, lower alkyl, amino, lower alkylamino, —CO₂H, and —CO₂(lower alkyl); or a progdrug carbamate thereof wherein at least one of R₁ and R₂ is —COOR, wherein R is hydrogen, C₁₋₆alkyl, benzyl, or —CH(OCOCH₃)CH₃, or a pharmaceutically-acceptable salt or hydrate of said compound or prodrug carbamate; wherein the compound has an IC₅₀ for inhibiting Factor XIa of less than 20 nM.

Most preferred methods comprise administering to a mammal a pharmaceutical composition containing a compound having the formula (Ib),

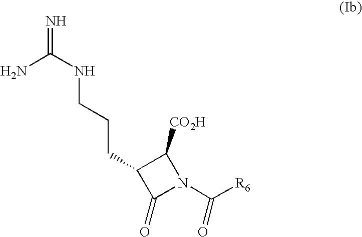

wherein:

R₆ is

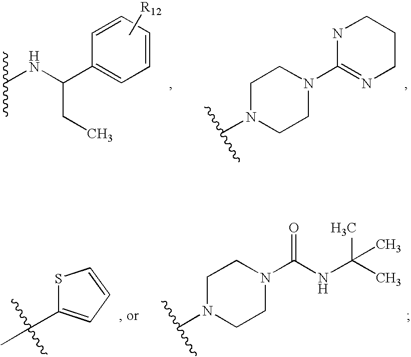

or a progdrug carbamate thereof wherein at least one of R₁ and R₂ is —COOR, wherein R₁₂ is defined as above; R is hydrogen, C₁₋₆alkyl, benzyl, or —CH(OCOCH₃)CH₃, or a pharmaceutically-acceptable salt or hydrate of said compound or prodrug carbamate; wherein the compound has an IC₅₀ for inhibiting Factor XIa of less than 3 nM.

### UTILITY

The methods of the present invention are useful for treating or preventing those conditions which involve the action of Factor XIa. Accordingly, the methods of the present invention are useful in treating consequences of atherosclerotic plaque rupture including cardiovascular diseases associated with the activation of the coagulation cascade in thrombotic or thrombophilic states. As used herein, the terms “treating” or “treatment” encompass responsive and/or prophylaxis measures, e.g., measures designed to inhibit or delay the onset of the disease, achieve a full or partial reduction of the symptoms or disease state, and/or to alleviate, lessen, or cure the disease or disorder and/or its symptoms.

More particularly, the methods of the present invention may be used to treat acute coronary syndromes such as coronary artery disease, myocardial infarction, unstable angina (including crescendo angina), ischemia (e.g., ischemia resulting from vascular occlusion), and cerebral infarction. The methods of the present invention further may be useful in treating stroke and related cerebral vascular diseases (including cerebrovascular accident and transient ischemic attack); venous thrombosis and thrombo-embolism, such as deep vein thrombosis (DVT) and pulmonary embolism; thrombosis associated with atrial fibrillation, ventricular enlargement, dilated cardiac myopathy, or heart failure; peripheral arterial disease and intermittent claudication; the formation of atherosclerotic plaques and transplant atherosclerosis; restenosis following arterial injury induced endogenously (by rupture of an atherosclerotic plaque), or exogenously (by invasive cardiological procedures such as vessel wall injury resulting from angioplasty); disseminated intravascular coagulopathy, Kasabach-Merritt syndrome, cerebral thrombosis, cerebral embolism, and disseminated intravascular coagulopathy.

Additionally, the methods of the present invention may be useful in treating thrombo-embolic consequences or complications associated with surgery (such as hip replacement, endarterectomy, introduction of artificial heart valves, vascular grafts, mechanical organs, and implantation or transplantation of organ, tissue or cells); medications (such as oral contraceptives, hormone replacement, and heparin, e.g., for treating heparin-induced thrombocytopenia); and pregnancy or childbirth. The methods of the present invention may be used to treat thrombosis due to confinement (i.e. immobilization, hospitalization, bed rest, limb immobilization, e.g., with immobilizing casts, etc.).

The methods of the present invention also may be useful in preventing thrombosis and complications in patients genetically predisposed to arterial thrombosis or venous thrombosis (including activated protein C resistance, FV_{leiden}, Prothrombin 20210, elevated coagulation factors FVII, FVIII, FIX, FX, FXI, prothrombin, TAFI and fibrinogen), elevated levels of homocystine, and deficient levels of antithrombin, protein C, and protein S. The inventive methods may be used for treating heparin-intolerant patients, including those with congenital and acquired antithrombin III deficiencies, heparin-induced thrombocytopenia, and those with high levels of polymorphonuclear granulocyte elastase. The methods of this invention may be used to treat all forms of thrombophilia.

The methods of the present invention may also be used to maintain blood vessel patency, for example, in patients undergoing transluminal coronary angioplasty, or in connection with vascular surgery such as bypass grafting, arterial reconstruction, atherectomy, vascular grafts, stent patency, and organ, tissue or cell implantation and transplantation. The inventive methods may be used to inhibit blood coagulation in connection with the preparation, storage, fractionation, or use of whole blood. For example, the inventive methods may be used in maintaining whole and fractionated blood in the fluid phase such as required for analytical and biological testing, e.g., for ex vivo platelet and other cell function studies, bioanalytical procedures, and quantitation of blood-containing components, or for maintaining extracorpeal blood circuits, as in dialysis or surgery (e.g., coronary artery bypass surgery).

In addition, the methods of the present invention may be useful in treating and preventing the prothrombotic complications of cancer. The methods may be useful in treating tumor growth, as an adjunct to chemotherapy, for preventing angiogenesis, and for treating cancer, more particularly, cancer of the lung, prostate, colon, breast, ovaries, and bone.

The methods of the present invention also may be used to treat diabetes mellitus, hypertension, or hypercholesterolemia.

In carrying out the methods of the present invention, it may be desired to administer the small molecule Factor XIa inhibitors in combination with each other and one or more other agents for achieving a therapeutic benefit such as antithrombotic or anticoagulant agents, anti-hypertensive agents, anti-ischemic agents, anti-arrhythmic agents, platelet function inhibitors, and so forth. More particularly, the inventive methods may be carried out by administering the small molecule Factor XIa inhibitors in combination with aspirin, clopidogrel, ticlopidine or CS-747, warfarin, low molecular weight heparins (such as LOVENOX), GPIIb/GPIIIa blockers, PAI-1 inhibitors such as XR-330 and T-686, P2Y1 and P2Y12 receptor antagonists; thromboxane receptor antagonists (such as ifetroban), prostacyclin mimetics, thromboxane A synthetase inhibitors (such as picotamide), serotonin-2-receptor antagonists (such as ketanserin); compounds that inhibit other coagulation factors such as FVII, FVIII, FIX, FX, prothrombin, TAFI, and fibrinogen, and/or other compounds that inhibit FXI; fibrinolytics such as TPA, streptokinase, PAI-1 inhibitors, and inhibitors of α-2-antiplasmin such as anti-α-2-antiplasmin antibody fibrinogen receptor antagonists, hypolipidemic agents, such as HMG-CoA reductase inhibitors (e.g., pravastatin, simvastatin, atorvastatin, fluvastatin, cerivastatin, AZ4522, and itavastatin), and microsomal triglyceride transport protein inhibitors (such as disclosed in U.S. Pat. Nos. 5,739,135, 5,712,279 and 5,760,246); antihypertensive agents such as angiotensin-converting enzyme inhibitors (e.g., captopril, lisinopril or fosinopril); angiotensin-II receptor antagonists (e.g., irbesartan, losartan or valsartan); ACE/NEP inhibitors (e.g., omapatrilat and gemopatrilat); and/or β-blockers (such as propranolol, nadolol and carvedilol). The inventive methods may be carried out by administering the small molecule Factor XIa inhibitors in combination with anti-arrhythmic agents such as for atrial fibrillation, for example, amiodarone or dofetilide.

In carrying out the methods of the present invention, it may be desired to administer the small molecule Factor XIa inhibitors in combination with agents that increase the levels of cAMP or cGMP in cells for a therapeutic benefit. For example, the compounds of the invention may have advantageous effects when used in combination with phosphodiesterase inhibitors, including PDE1 inhibitors (such as those described in Journal of Medicinal Chemistry, Vol. 40, pp. 2196-2210 [1997]), PDE2 inhibitors, PDE3 inhibitors (such as revizinone, pimobendan, or olprinone), PDE4 inhibitors (such as rolipram, cilomilast, or piclamilast), PDE7 inhibitors, or other PDE inhibitors such as dipyridamole, cilostazol, sildenafil, denbutyline, theophylline (1,2-dimethylxanthine), ARIFLO™ (i.e., cis-4-cyano-4-[3-(cyclopentyloxy)-4-methoxyphenyl]cyclohexane-1-carboxylic acid), arofyline, roflumilast, C-11294A, CDC-801, BAY-19-8004, cipamfylline, SCH351591, YM-976, PD-189659, mesiopram, pumafentrine, CDC-998, IC-485, and KW-4490.

The inventive methods may be carried out by administering the Factor XIa inhibitors in combination with prothrombolytic agents, such as tissue plasminogen activator (natural or recombinant), streptokinase, reteplase, activase, lanoteplase, urokinase, prourokinase, anisolated streptokinase plasminogen activator complex (ASPAC), animal salivary gland plasminogen activators, and the like.

The inventive methods may be carried out by administering the Factor XIa inhibitors in combination with β-adrenergic agonists such as albuterol, terbutaline, formoterol, salmeterol, bitolterol, pilbuterol, or fenoterol; anticholinergics such as ipratropium bromide; anti-inflammatory cortiocosteroids such as beclomethasone, triamcinolone, budesonide, fluticasone, flunisolide or dexamethasone; and anti-inflammatory agents such as cromolyn, nedocromil, theophylline, zileuton, zafirlukast, monteleukast and pranleukast.

The small molecule Factor XIa inhibitors may act synergistically with one or more of the above agents. Thus, reduced doses of thrombolytic agent(s) may be used, therefore obtaining the benefits of administering these compounds while minimizing potential hemorrhagic and other side effects.

In practicing the methods of the present invention, the small molecule Factor XIa inhibitors, such as compounds of formula (I), may be administered by any means suitable for the condition to be treated, which may depend on the need for site-specific treatment or quantity of drug to be delivered. The compounds may be delivered orally, such as in the form of tablets, capsules, granules, powders, or liquid formulations including syrups; sublingually; bucally; transdermally; parenterally, such as by subcutaneous, intravenous, intramuscular or intrastemal injection or infusion (e.g., as sterile injectable aqueous or non-aqueous solutions or suspensions); nasally such as by inhalation spray; rectally such as in the form of suppositories, or in the form of liposome particles. Dosage unit formulations containing non-toxic, pharmaceutically acceptable vehicles or diluents may be administered. The compounds may be administered in a form suitable for immediate release or extended release. Immediate release or extended release may be achieved with suitable pharmaceutical compositions or, particularly in the case of extended release, with devices such as subcutaneous implants or osmotic pumps.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The inventive compounds may be orally delivered by sublingual and/or buccal administration, e.g., with molded, compressed, or freeze-dried tablets. Exemplary compositions may include fast-dissolving diluents such as mannitol, lactose, sucrose, and/or cyclodextrins. Also included in such formulations may be high molecular weight excipients such as celluloses (AVICEL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®); and agents to control release such as polyacrylic copolymer (e.g., CARBOPOL 934®). Lubricants, glidants, flavors, coloring agents and stabilizers may also be added for ease of fabrication and use.

Exemplary compositions for nasal aerosol or inhalation administration include solutions which may contain, for example, benzyl alcohol or other suitable preservatives, absorption promoters to enhance absorption and/or bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, suitable non-irritating excipients, such as cocoa butter, synthetic glyceride esters or polyethylene glycols, which are solid at ordinary temperatures but liquefy and/or dissolve in the rectal cavity to release the drug.

The effective amount of a small molecule Factor XIa inhibitor administered according to the present invention may be determined by one of ordinary skill in the art. The specific dose level and frequency of dosage for any particular subject may vary and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition. An exemplary effective amount of compounds of formula (I) may be within the dosage range of about 0.1 to about 100 mg/kg, preferably about 0.2 to about 50 mg/kg and more preferably about 0.5 to about 25 mg/kg (or from about 1 to about 2500 mg, preferably from about 5 to about 2000 mg) on a regimen in single or 2 to 4 divided daily doses.

### ASSAYS

Assays were administered for determining Factor FXIa activity, selectivity and comparative selectivity, clotting time and platelet function, in vivo efficacy and safety, bleeding and coagulatoin times, as described below.

### COMPOUND PREPARATION

For in vitro studies, the compounds were dissolved in DMSO to give concentrated stock solutions of 5 mM. The stocks were subsequently diluted in DMSO. For enzyme assays, 2 μL was added to a total 300 μL assay volume. For in vivo studies, compound was prepared in saline at a concentration of up to 12 mg/mL.

### FXIA ENZYMATIC ACTIVITY

Human FXIa activity (Enzyme Research Labs or Haematologic Technologies Inc.) was measured at room temperature in 96 well microtiter plates. The enzyme was incubated with drug for 3 minutes at an enzyme concentration of 0.5 nM in 145 mM NaCl, 5 mM KCl, 1 mg/mL PEG 8000,30 mM HEPES (pH 7.4). After the three minute incubation, 100 μM substrate, S-2366, (L-Pyroglut-Pro-Arg-pNA, Diapharma Group, Inc.) was added and the enzyme velocity was measured using a Molecular Devices Spectro Max Plus (405 nm wavelength) in kinetic mode and analyzed using SOFTmax Pro (ver 3.0) software. For routine compound evaluation, the fraction of control activity (FCA) was plotted as a function of the inhibitor concentration (I), and the curve was fit to the equation FCA /(1+[I]/IC₅₀) to determine the IC₅₀.

A fluorescent assay was performed under similar conditions to evaluate FXIa activity of compounds with high potency (IC₅₀<10 nM). A different substrate (D-Ile-Pro-Arg-AFC.2TFA) and a different concentration of enzyme (0.043 nM) was used. The enzyme velocity was measured using a Molecular Devices fMax fluorescent plate reader (400 nm excitation, 505 nm emission).

### SELECTIVITY ASSAYS

Enzymatic activity of human α-thrombin (Sigma) was measured in a buffer containing 0.145 M NaCl, 0.005 M KCl, 1 mg/mL polyethylene glycol (PEG-8000), 0.030 M HEPES (pH 7.4), and 0.03 U/mL final thrombin concentration using a microtiter plate-based assay. The enzyme was incubated at room temperature with the inhibitor for 3 min prior to starting the reaction with 10 μM S-2238 (D-Phe-Pip-Arg-pNA; Km=2.54 μM).

Plasmin (Kabi Vitrum) was measured as for thrombin except that the buffer was 50 mM TRIS/Cl (pH 7.8) and the reaction was initiated with 100 μM S-2251 (D-Val-Leu-Lys-pNA; Km=98 μM).

Recombinant tissue plasminogen activator (Genentech), human factor Xa (Kabi Vitrum), and urokinase (Abbott Labs, Abbokinase) were assayed in the same buffer as thrombin, but the reactions were started with 100 μM spectrozyme t-PA (methylsulfonyl-D-cyclohexyltyrosyl-Gly-Arg-pNA; Km=90 μM), 100 μM S-2222 (phenyl-Ile-Glu-Gly-Arg-pNA; Km=87 μM), and 100 μM S-2444 (L-pyroGlu-Gly-Arg-pNA; Km=31 μM), respectively.

FXIIa (American Diagnostica) catalytic activity was measured in 150 mM NaCl, 50 mM Tris, 50 mM Imidazole, pH 8.2 using 100 μM spectrozyme FXIIa (D-cyclohexyltyrosyl-Gly-L-Arg-pNA; Km=40.2 μM).

FIXa (American Diagnostica) activity was measured in 100 mM NaCl, 5 mM CaCl₂, 33% ethylene glycol, 50 mM Tris, pH 7.5 with 100 μM spectrozyme FIXa (MeSO₂-D-cyclohexylglycyl-Gly-L-Arg-pNA; Km>100 μM).

Human TF:FVIIa activity was measured using isolated human FVIIa (Enzyme Research Labs) in the presence of recombinant human tissue Factor (Dade Innovin) in a buffer containing 20 mM HEPES, 150 mM NaCl, 5mM CaCl₂, 1 mM CHAPS and 1 mg/mL PEG 6000 (pH to 7.4). After a three minute incubation with the drug, 100 μM substrate S-2288 (Km>500μM) was added and optical densitity was recorded over time in the microplate reader.

### CLOTTING TIME AND PLATELET FUNCTION ASSAYS

PT and APTT clotting times were determined as described in Balasubramanian et al., J. Med. Chem., Vol. 36 (1993), at pp. 300-03, incorporated herein by reference. The whole blood aggregation response of rat platelets to collagen was determined using the impedance procedure for a Model 560-CA Chrono-Log aggregometer (Havertown, Pa.).

### IN VIVO EFFICACY AND SAFETY ASSAYS FOR THROMBOSIS INHIBITION

General Surgical Preparation Male Sprague Dawley rats (310 to 390 g) were anesthetized with Na-pentobarbital (50 mg/kg i.p.). The left jugular vein was cannulated with PE-50 tubing for drug administration, and the trachea was intubated with PE-205 tubing to ensure airway patency. In some animals, a carotid artery was cannulated with PE-50 tubing to obtain blood samples, and a procedure was followed as described in Schumacher et al., “Comparison of Thrombin Active-Site and Exosite Inhibitors and Heparin in Experimental Models of Arterial and Venous Thrombosis and Bleeding,” J. Pharmacol. Exp. Therap., Vol. 267 (1993), at pp. 1237-42; Schumacher et al., Effects of Antithrombotic Drugs in a Rat Model of Aspirin-Insensitive Arterial Thrombosis, Thromb Haemostasis, Vol. 69 (1993), at pp. 509-514; or Schumacher et al., Effect of a Novel Thrombin Active-Site Inhibitor on Arterial and Venous Thrombosis, Eur J Pharmacol., Vol. 259 (1994), at pp. 165-171, each of which is incorporated herein by reference.

In each procedure, a small organic compound effective for inhibiting Factor XIa, as described herein, or saline vehicle, was administered i.v. as a loading infusion over 5 min plus a sustaining infusion that was maintained until the end of the procedure. Compound doses are indicated for each procedure in mg/kg plus mg/kg/hr. The volume of test article was 1 mL/kg plus 1 mL/kg/hr, except the 24 mg/kg+24 mg/kg/hr dose of compound where volume was doubled.

Arterial Thrombosis The left carotid artery was exposed and a piece of parafilm “M” (American National Can, Greenwich, Conn.) was inserted under the vessel. An electromagnetic flow probe (1.0 mm lumen) was placed on the artery and attached to a model MDL 1401 flowmeter (Skalar, Delft, Netherlands). Following baseline flow measurements, a 2-mm by 5-mm strip of filter paper saturated with a 50% solution of FeCl₂ was placed on top of the vessel downstream from the flow probe for a period of 10 min. The carotid artery was removed at 60 min after filter paper application, and the thrombus isolated and weighed immediately using a AE50 balance (Mettler, Toledo, Ind.). Carotid blood flow (CBF) was monitored continuously on a TA3800 physiologic recorder (Gould, Cleveland, Ohio). Total CBF was determined and normalized as percent of baseline (0 min) flow over 60 min to provide a measure of average blood flow during thrombus formation.

Venous Thrombosis Two venous thrombosis (VT) models were used, namely, a vessel injury/topical FeCl₂ model and a stasis VT model.

In the vessel injury model, the vena cava was isolated via a midline abdominal incision and the surface cleared by blunt dissection between the renal and illiolumbar veins. A 2-mm by 5-mm strip of filter paper saturated with 15% FeCl₂ was placed on the vena cava for 1 min. Sixty min after filter paper application, the vena cava was dissected free, and the thrombus was removed and weighed immediately using a Mettler AE50 balance.

In the stasis model, the vena cava was isolated via a midline abdominal incision. A vena cava sac was produced by tying a ligature around a blunted 26-gauge needle just distal to the renal veins and applying a microaneurysm clamp just proximal to the bifurcation of the femoral veins. A separate 26-gauge hypodermic needle was inserted into the inferior portion of the venous sac, and hypotonic saline (0.225% NaCl) was infused at a rate of 10 ml/min for 15 sec. The injection needle was removed following the saline flush and the hole was sealed with cyanoacrylate cement. The proximal needle was then slipped free from the ligature, leaving a fixed nonocclusive stenosis, and the distal vascular clamp was removed. After maintaining blood flow through the stenosis for 20 min, the caval sac was re-isolated and dissected free. The thrombus was removed and weighed immediately on the Mettler balance.

Experimental Bleeding Time Two models of bleeding time were performed, namely, a renal cortex bleeding model (using template incision of the renal cortex), and a mesenteric artery bleeding model (using severance of small mesenteric arteries).

In the renal cortex bleeding time (RCBT) model, both kidneys were exposed by a midline abdominal incision, and the renal capsules were removed to expose the bare renal cortex which was superfused with Ringer's solution maintained at 37° C. Controlled incisions were created using a Surgicutt template device (International Technidyne Corp., Edison, N.J.), which produced a 5-mm long by 1-mm deep cut with the tip of a spring-loaded surgical blade (No. 25, sharp tip). While observing under 3×-binocular magnifier, the time in sec from injury until bleeding stopped was recorded. Three bleeding times were determined on the right kidney before, and on the left kidney 15 min after, the start of test article infusion. Replicate bleeding times were averaged to single values for statistical comparison.

In the mesenteric artery bleeding time (MABT) model, the abdomen was opened via a midline incision and the small intestine was exteriorized. The jejunum was exposed, held in place with clamps and superfused with Ringer's solution maintained at 37° C. Small arteries that branch perpendicular to the mesenteric artery and course over the surface of the jejunum were observed through an SZH10 stereomicroscope (Olympus Corp., Lake Success, N.Y.). These vessels were punctured with a 30-gauge hypodermic needle and the time in sec from puncturing until bleeding stopped was recorded. Bleeding times were determined in 3-5 arteries before administration of test article and in 3-5 arteries 20 min after the start of test article infusion. Replicate bleeding times were averaged to single values for statistical comparison.

Ex Vivo Clotting Times Ex vivo clotting time assays were used to determine the level of anticoagulation achieved in the thrombosis and bleeding time procedures, described above. A small organic compound for inhibiting Factor XIa was administered at doses (mg/kg+mg/kg/hr) of 0.5+0.5 (n=3), 2+2 (n=4), 6+6 (n=3), 12+12 (n=3) and 24+24 (n=10). Carotid artery blood (0.6 mL drawn into 1/10 volume of 3.8% Na-citrate) was sampled before (0 min control) and at 5, 15, 30, 60 and 90 min after the start of the compound infusion. The APTT was determined in freshly prepared plasma from each sample using the standard procedure for Dade Actin FSL reagent (Baxter Healthcare Corp., Miami, Fla.). The PT was determined in some animals from the 6+6 (n=3), 12+12 (n=3) and 24+24 (n=2) dose groups using Dade Thromboplastin●C reagent.

Statistical Analysis In the venous and arterial thrombosis procedures, treatment effects on thrombus weight, baseline blood flow, and average blood flow during thrombosis were determined by analysis of variance with Dunnett's test for comparison to the vehicle group. The Fisher exact test was used to compare frequency of occlusion data between treatment groups. Treatment effects on bleeding times were determined by analysis of variance with Dunnett's test for comparison to the vehicle group. Both post-treatment and pre-treatment bleeding times and the relative bleeding time increases in sec were analyzed. Computations were performed using Systat software (Evanston, Ill.). Data are presented as mean ±SEM. A p<0.05 was considered significant.

The following Examples are set forth to illustrate the invention, without limiting the scope thereof.

### EXAMPLES

Compounds shown in Examples 1-15 below were each tested in the above described Factor XIa assay and discovered by the instant applicants to be surprisingly potent and selective inhibitors of Factor XIa.

### Example 1

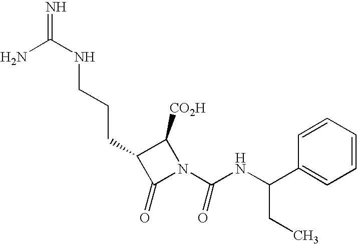

The compound of Example 1 was prepared as set forth in U.S. Pat. No. 6,335,324, which is incorporated herein by reference. Applicants herein discovered that Example 1, was a surprisingly highly potent inhibitor of FXIa with an IC₅₀ of approximately 100 pM.

### Examples 2-7

Each of the compounds of Examples 2-7 in Table 1 below, prepared as set forth in U.S. Pat. No. 6,335,324 (incorporated herein), demonstrated an inhibitory effect for Factor XIa with an IC₅₀ of less than or equal to about 1 to 6 nM.

TABLE 1

| Ex. | | IC₅₀(≅)(nM) |
|---|---|---|
| 2 | 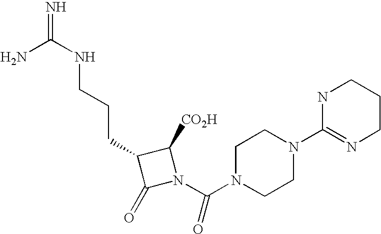 | 1 |
| 3 | 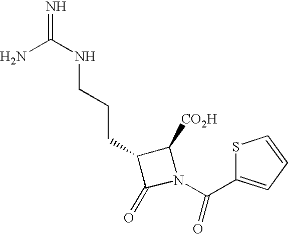 | 2 |
| 4 | 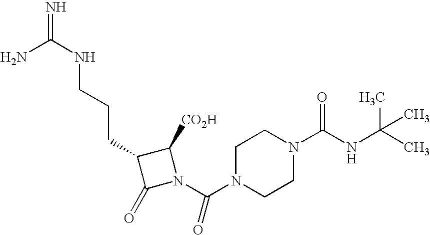 | 2.8 |
| 5 | 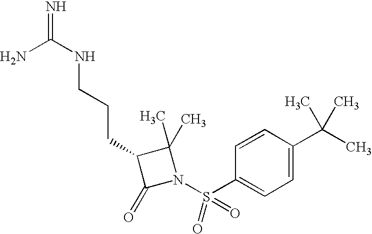 | 6 |
| 6 | 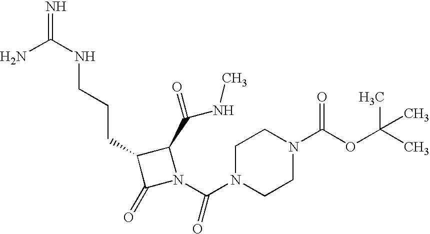 | |
| 7 | 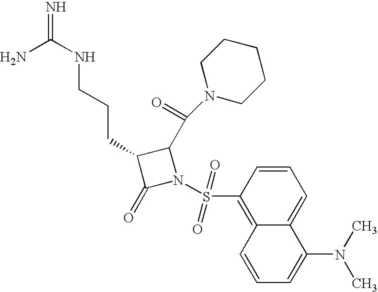 | 6 |

### Examples 8-15

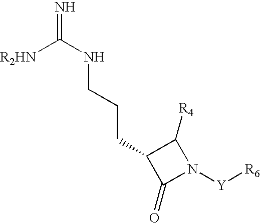

Each of the compounds of Examples 8-15, wherein R₂, R₄, Y and R₆ have the values listed in Table 2, below, (prepared as set forth in U.S. Pat. No. 6,335,324, incorporated herein), demonstrated an inhibitory effect for Factor XIa with an IC₅₀ of less than 120 nM.

TABLE 2

| Ex. No. | R₂ | R₄ | Y | R₆ | IC₅₀ (nM) |
|---|---|---|---|---|---|
| 8 | H | CO₂H | C(═O) | t-Bu | 12 |
| 9 | H | 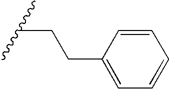 | C(═O) | CH₃ | 16 |
| 10 | H | CO₂H | C(═O) | 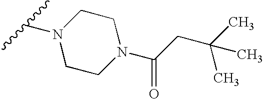 | 16 |
| 11 | H | CO₂H | SO₂ | 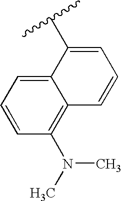 | 24 |
| 12 | CO₂Bu | 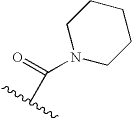 | C(═O) | CH₃ | 34 |
| 13 | H | CO₂H | C(═O) | CH₃ | 77 |
| 14 | H | 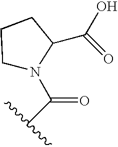 | C(═O) | CH₃ | 105 |
| 15 | H | CO₂H | C(═0) | t-Bu | 111 |

### Example 16

### Selectivity for Inhibition of Factor XIa

Small organic compounds described in the above Examples are comparatively selective for inhibition of Factor XIa as compared with “direct acting” coagulation protease inhibitors, i.e., active-site inhibitors of thrombin and Factor Xa. For example, the compound of Example 4 and compounds 16A and 16B, below, known active-site inhibitors of thrombin and Factor Xa, respectively, were tested in assays (described above) for their effects in inhibiting the activity of thrombin, plasmin, recombinant tissue plasminogen activator, human factor Xa, urokinase, FXIIa, FIXa, and human TF:FVIIa. The results are reported below in Table 3.

Compound 16A:

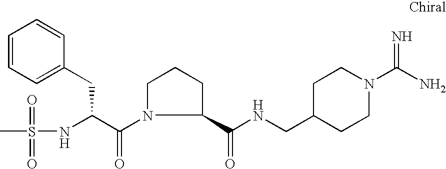

(See U.S. Pat. No. 5,583,146, to Bristol-Myers Squibb Company, incorporated herein by reference.)

Compound 16B:

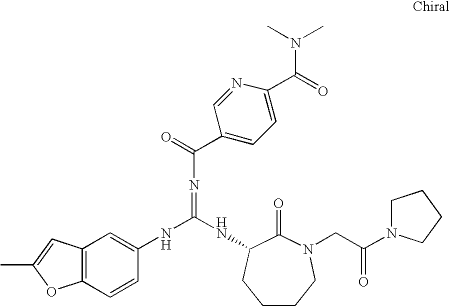

(See WO 00/47207, to Bristol-Myers Squibb Company, incorporated herein by reference.)

TABLE 3

| | | Thrombin | |
|---|---|---|---|
| | FXIa inhibitor | Inhibitor | FXa inhibitor |
| | (Example 4) | (Compound 16A) | (Compound 16B) |
| Enzyme | IC₅₀ (μM) | IC₅₀ (μM) | IC₅₀ (μM) |
| TF: FVIIa | 0.486 | 20 | >33 |
| FXIIa | >33 | >33 | 14.2 |
| FXIa | 0.0028 | 6.86 | >33 |
| FIXa | 17.4 | >33 | >33 |
| FXa | >33 | >333 | 0.00895 |
| Thrombin | 10.5 | 0.046 | 11.4 |
| Plasmin | 1.7 | 48 | >33 |
| Urokinase | 0.542 | >333 | >33 |
| Tissue | >33 | 95 | 1.44 |
| plasminogen | | | |
| activator | | | |

As can be seen from Table 3, Example 4 was greater than 2,000 times more active than the thrombin inhibitor (compound 16A), and more than 10,000 times more active than the FXa inhibitor, for inhibiting Factor XIa. Example 4 was also tested against a panel of 40 receptors and enzymes (MDS Panlabs Pharmacology Services). No significant inhibition or enhancement of receptor and enzyme activities was observed. The small organic compounds described herein are each highly selective and potent for inhibition of Factor XIa as compared with other blood coagulation factors and fibrinolysis proteases, such as FII, FIIa, FVII, FVIIa, FVIX, FVIXa, FX, FXa, tissue factor, fibrin, fibrinogen, and/or thrombin.

### Example 17

### PT and APTT Clotting Times

The compound of Example 4 and compounds 16A and 16B were tested in PT and APTT assays, described above. In human plasma, compound of Example 4 doubled the APTT at 0.6 μM, but did not prolong the conventional prothrombin time at the highest concentration (100 μM) tested. Similarly, using rat plasma, compound of Example 4 doubled the APTT at 2.1 μM but did not affect the PT at concentrations of up to 100 μM. Accordingly, Example 4 is selective for inhibiting the intrinsic cascade as compared with the extrinsic cascade. In comparison, compounds 16A (the thrombin inhibitor) and 16B (the FXa inhibitor), prolonged both the PT as well as APTT. These results are reported below in Table 4.

TABLE 4

| | FXIa inhibitor | Thrombin Inhibitor | FXa inhibitor |
|---|---|---|---|
| Clotting time | (Example 4) | (Compound 16A) | (Compound 16B) |
| (species) | (μM) | (μM) | (μM) |
| PT | >100 | 0.30 | 2.5 |
| (human) | | | |
| APTT | 0.6 | 0.15 | 4.0 |
| (human) | | | |
| PT | >100 | 0.24 | 2.8 |
| (rat) | | | |
| APTT | 2.1 | 0.29 | 4.5 |
| (rat) | | | |

### Example 18

### In Vivo Efficacy of Factor XIa Inhibitor for Inhibiting Arterial Thrombosis

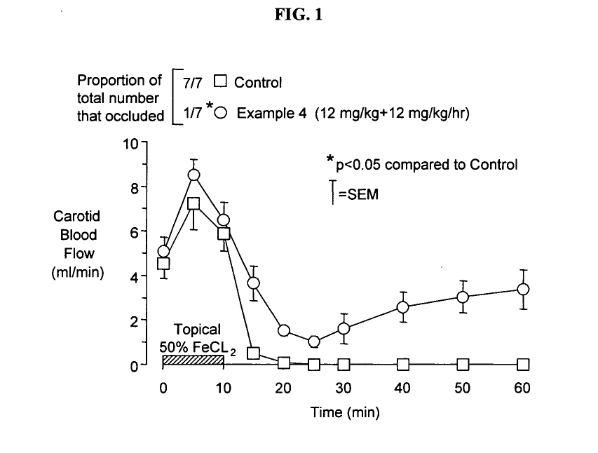

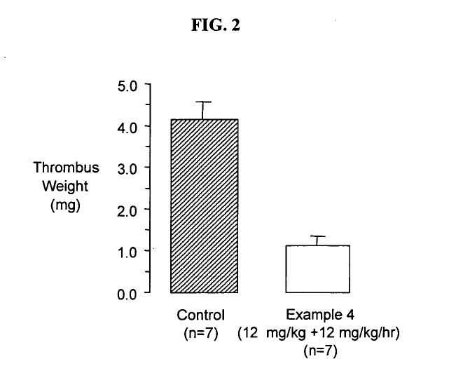

The compound of Example 4 was administered via an intravenous infusion into six rats following the arterial thrombosis model described above. Carotid artery injury with FeCl₂ was initiated 10 min after the start of test article infusion, which was maintained until thrombus removal. Intravenous infusion of the compound at a dose of 12 mg/kg plus 12 mg/kg/hr prevented FeCl₂-induced carotid artery thrombosis as illustrated in FIG. 1. Additionally, the compound caused a 73% decrease in thrombus weight, as illustrated in FIG. 2. There was also a concurrent improvement in both average blood flow during thrombosis and vessel patency at this dose of compound.

### Example 19

### In Vivo Efficacy of Factor XIa Inhibitor for Inhibiting Venous Thrombosis

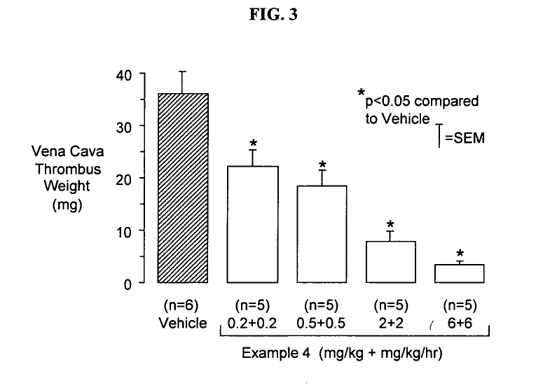

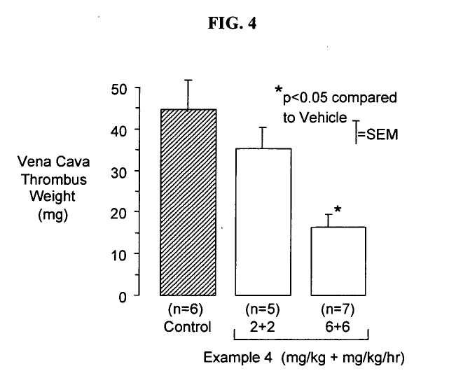

The compound of Example 4 was tested in two models for inhibiting venous thrombosis, i.e., the vessel injury model and statis model, described above. In the vessel injury model, the compound was administered at i.v. doses (mg/kg+mg/kg/hr) of 0.2+0.2 (n=5), 0.5+0.5 (n=5), 2+2 (n=5), 6+6 (n=5). There were six vehicle-treated rats in each model. Thrombosis was initiated 10 min after the start of test article infusion which was maintained until thrombus removal. The results are reported in FIG. 3. As can be seen, the compound was efficacious in rat models of VT induced by severe oxidative vessel injury (FeCl₂-VT). Dose-dependent reductions in thrombus weight were observed. A reduction of approximately 90% was achieved at a 6+6 dose (mg/kg+mg/kg/hr), while a 30-fold lower dose of 0.2+0.2 still produced a significant inhibition of about 38%.

In the stasis VT model, the compound was administered at i.v. doses (mg/kg+mg/kg/hr) of 2+2 (n=5) and 6+6 (n=5). The compound produced a 64% reduction in thrombus weight at a 6+6 dose (mg/kg+mg/kg/hr). However, little effect was observed with the 2+2 dose that had been effective in FeCl₂-VT. Accordingly, applicants have discovered that the Factor XIa inhibitor has greater efficacy in the vessel-injury model of VT than the stasis model. In comparison, thrombin, FXa inhibitors, and Warfarin are more efficacious in the stasis model than vessel injury model, and antiplatelet drugs tend to be more active in the FeCl₂-VT model compared to stasis-VT model.

### Example 20

### Bleeding Times and Comparative Analysis

The compound of Example 4 was tested in experimental models for determining renal cortex bleeding times (RCBT) and mesenteric artery bleeding times (MABT), as described above. These bleeding time models are sensitive to both antiplatelet (aspirin) and anticoagulant drugs (heparin, LMWH, thrombin inhibitors and FXa inhibitors), and show whether antithrombotic activity could be dissociated from effects on haemostasis.

As described above, bleeding times were determined in pentobarbital-anesthetized rats by template incision of the renal cortex or by puncturing mesenteric arteries that course over the surface of the jejunum with a hypodermic needle. In each rat 3 to 5 replicate bleeding times were performed before (pre-treatment) and 15 min after (post-treatment) i.v. infusion of either saline vehicle or test compound. In both models, infusion of test article was maintained until bleeding time measurements were finished (˜30 min total infusion). The compound of Example 4 was administered at doses (mg/kg+mg/kg/hr) of 12+12 and 24+24 in both models, wherein in the renal model (n=4 each dose) and the mesenteric model (n=5 and n=4, respectively). Four rats were treated in the RCBT model and five in the MABT model.

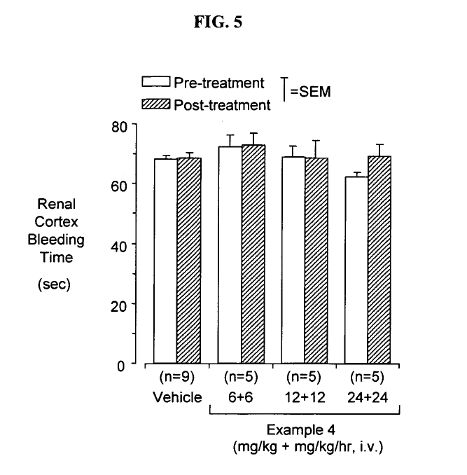

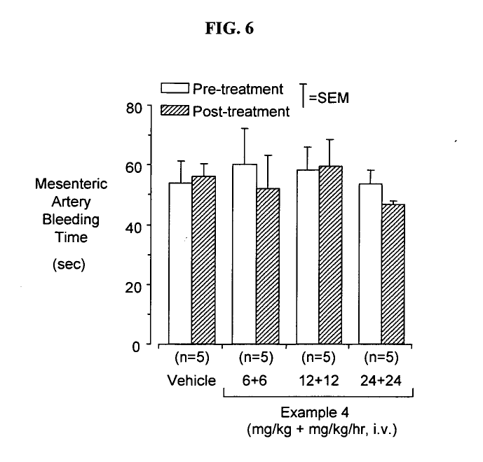

The results of these models are reported in FIGS. 5 and 6, which are bar graphs showing the renal cortex and mesenteric artery bleeding times pre- and post-treatment upon administration of the test compound. As can be seen, when given at doses that equaled and exceeded maximum antithrombotic doses, the compound had no effect on renal cortex bleeding times (measured using template incisions) or a mesenteric artery bleeding times. The small organic compound Factor XIa inhibitor did not affect renal cortex or mesenteric artery bleeding times when given at doses that were two times those required for optimal antithrombotic activity.

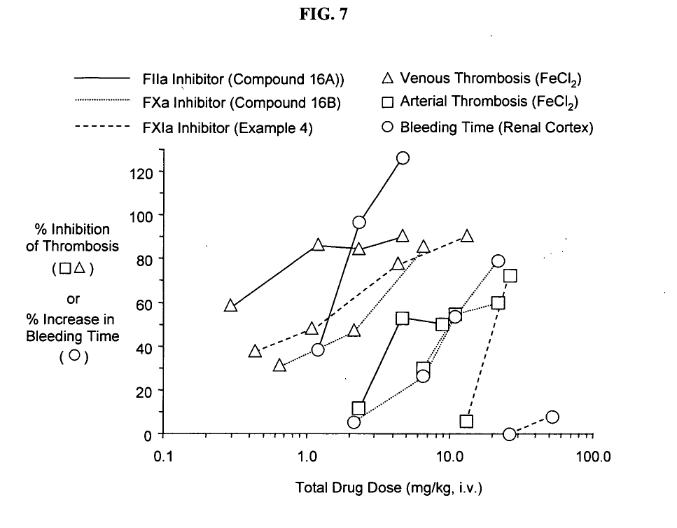

FIG. 7 reports an analysis of the differential effect on thrombosis inhibition and and bleeding time prolongation in anesthetized rats for the Factor XIa inhibitor, as compared with the “direct acting” thrombin and FXa active site inhibitors, namely, compounds 16A and 16B (thrombin and FXa active site inhibitors). The thrombin inhibitor (open symbols), FXa inhibitor (dotted symbols) and FXIa inhibitor (line symbols) were tested for effects on arterial thrombosis (squares), venous thrombosis (triangles) and bleeding time (circles) in rat models. Compounds were administered as loading plus sustaining infusion and the total dose is given on the x-axis. As can be seen, the FXIa inhibitor surprisingly shows a substantially greater separation between bleeding time prolongation and efficacy in venous and arterial thrombosis than the thrombin and Fxa inhibitors. These studies demonstrate that FXIa active site inhibition is a useful method for preventing arterial and venous thrombosis without incurring bleeding risk.

### Example 21

### Ex Vivo Anticoagulant Activity of Factor XIa Inhibitor in Rats

To determine the extent of systemic anticoagulation associated with antithrombotic activity, the compound of Example 4 was administered as a loading plus sustaining infusion in pentobarbital-anesthetized rats, at doses (mg/kg+mg/kg/hr) of 0.5+0.5 (n=3), 2+2 (n=4), 6+6 (n=3), and 12+12 (n=3). Plasma was sampled before (0 min control) and after the infusion, at intervals of 5, 15, 30, 60 and 90 min after initiating the infusion. Average APTT increases over the 15-90 min period, an interval most applicable to thrombosis and bleeding time protocols, were calculated. Control APTT values averaged 19.9±1.3 seconds overall. The PT was unaltered as expected from in vitro results. The APTT was prolonged, and the increase in APTT is reported in Table 5. As can be seen, the dose-response for this effect was essentially flat. The range of efficacious doses in venous and arterial thrombosis (2+2, 6+6, 12+12; mg/kg+mg/kg/hr) produced an APTT increase of about 2.8 times control.

TABLE 5

| Compound dose | APTT Increase |
|---|---|
| (mg/kg + mg/kg/hr) | (X Control) |
| 0.5 + 0.5 (n = 3) | 1.57 ± 0.22 |
| 2 + 2 (n = 4) | 2.81 ± 0.21 |
| 6 + 6 (n = 3) | 2.73 ± 0.19 |
| 12 + 12 (n = 3) | 2.79 ± 0.26 |
| 24 + 24 (n = 10) | 3.29 ± 0.85 |

We claim:

1. A method of treating thrombosis in a mammal comprising administering to the mammal a pharmaceutical composition that inhibits thrombosis in the mammal, wherein the pharmaceutical composition contains a therapeutically-effective amount of a small organic compound that is potent and selective for inhibiting Factor XIa so that the differential rate of percent inhibition of thrombosis in the mammal is greater than the differential rate of percent increase in bleeding time.

2. The method of claim 1, in which the differential rate of percent inhibition of thrombosis weight in the mammal is at least 50% while the differential rate of percent increase in bleeding time is not increased or just measurably increased by less than 30%.

3. The method of claim 1, in which the small organic compound has an IC₅₀ for inhibiting Factor XIa of less than 10 nM.

4. The method of claim 1, in which the small organic compound has an IC₅₀ for inhibiting Factor XIa of less than 6 nM.

5. The method of claim 1, in which the small organic compound has an IC₅₀ for inhibiting Factor XIa of less than 3 nM.

6. The method of claim 1, in which the small organic compound is highly selective for inhibiting Factor XIa.

7. A method of treating thrombosis in a mammal comprising administration of a small organic compound to the mammal having sufficient selectivity and potency for inhibition of Factor XIa, wherein the administration of the small molecule inhibits thrombosis in the mammal with no substantial effect on bleeding times in the mammal.

8. The method of claim 7, in which the small organic compound has an IC₅₀ for inhibiting Factor XIa of less than 6 nM.

9. The method of claim 7, in which the small organic compound has an IC₅₀ for inhibiting Factor XIa of less than 3 nM.

10. The method of claim 7, in which the small organic compound is highly selective for inhibiting Factor XIa.

11. A method of inhibiting Factor XIa in a mammal by administration of a small organic compound with an IC₅₀ for inhibiting Factor XIa of less than 120 nM.

12. The method of claim 11, wherein the small organic compound has an IC₅₀ for inhibiting Factor XIa of less than 10 nM.

13. The method of claim 11, wherein the small organic compound has an IC₅₀ for inhibiting Factor XIa of less than 6 nM.

14. The method of claim 11, wherein the small organic compound has an IC₅₀ for inhibiting Factor XIa of less than 1 nM.

15. A method of inhibiting Factor XIa in a mammal by administration of a small organic compound having the formula (I): 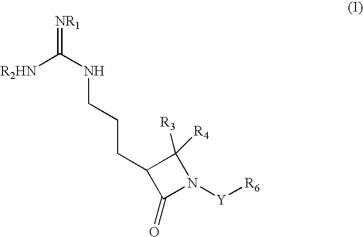
wherein:
R₁ and R₂ are hydrogen;
R₃ is hydrogen or CH₃;
R₄ is selected from hydrogen, CH₃, —CO₂R₇, —C(═O)NR₈R₉, phenyl, benzyl, and phenylethyl, wherein R₇ is hydrogen, C₁₋₆alkyl, benzyl, or —CH(OCOCH₃)CH₃; and each R₄ group is optionally substituted with one to two R₁₂;
Y is C(═O) or —SO₂—; wherein when Y is C(═O), then R₆ is C₁₋₆alkyl, aryl, heteroaryl, or —NR₁₀R₁₁, and when Y is —SO₂—, then R6 is aryl or heteroaryl; and each R₆ group is optionally substituted with one to two R₁₂;
R₈ and R₉ are individually selected from hydrogen and C₁₋₆alkyl, or R₈ and R₉ taken together form a five or six membered heterocyclo ring optionally substituted with one to two R₁₂ and up to one R₁₃;
R₁₀ and R₁₁ are individually selected from hydrogen, phenyl, or C₁₋₆alkyl optionally substituted with phenyl, or R₁₀ and R₁₁ taken together form a five or six membered heterocyclo ring optionally substituted with one to two R₁₂ and up to one R₁₃;
R₁₂ is selected from hydrogen, halogen, trifluoromethyl, trifluoromethoxy, lower alkyl, amino, lower alkylamino, —CO₂H, —CO₂(lower alkyl), or a five or six membered saturated or unsaturated heterocyclo having up to two nitrogen heteroatoms;
R₁₃ is selected from —C(═O)(C₁₋₆alkyl), —CO₂(C₁₋₆alkyl), —C(═O)NH(C₁₋₆alkyl), and five or six membered heterocyclo optionally substituted with one to two R₁₄; and
R₁₄ is selected from hydrogen, phenyl, or C₁₋₆alkyl optionally substituted with phenyl;
or a progdrug carbamate thereof wherein at least one of R₁ and R₂ is COOR, wherein R is hydrogen, C₁₋₆alkyl, benzyl, or CH(OCOCH₃)CH₃, or a pharmaceutically-acceptable salt or hydrate of said compound or prodrug carbamate.

16. The method of claim 15, wherein the small organic compound has the formula (Ia): 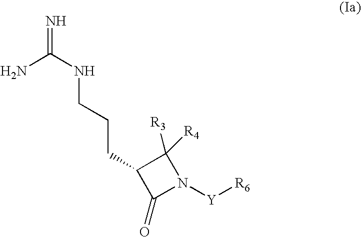
wherein:
R₃ is hydrogen or CH₃;
R₄ is CH₃, CO₂H, CO₂(C₁₋₄alkyl), 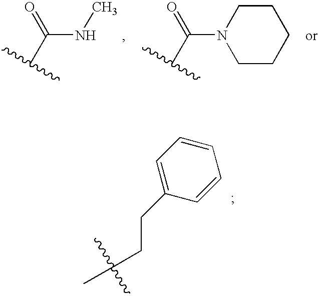
Y is C(═O) or —SO₂—; wherein:
when Y is C(═O), then R₆ is methyl, ethyl propyl,
when Y is —SO₂—, then R₆ is selected from 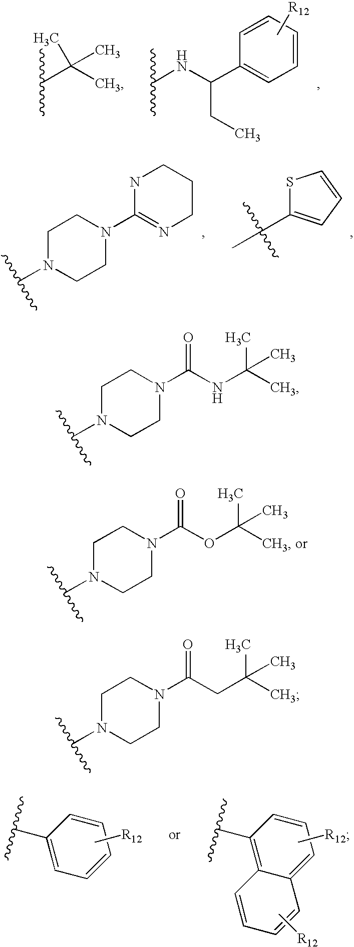
and
R₁₂ is selected from hydrogen, lower alkyl, amino, lower alkylamino, —CO₂H, and —CO₂(lower alkyl); or a progdrug carbamate thereof wherein at least one of R₁ and R₂ is —COOR, wherein R is hydrogen, C₁₋₆alkyl, benzyl, or —CH(OCOCH₃)CH₃, or a pharmaceutically-acceptable salt or hydrate of said compound or prodrug carbamate; wherein the compound has an IC₅₀ for inhibiting Factor XIa of less than 20 nM.

17. The method of claim 15, wherein the small organic compound has the formula (Ib), 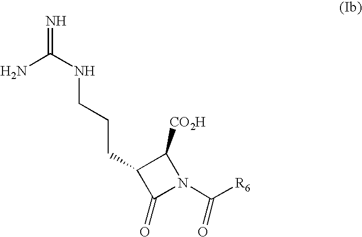
wherein:
R₆ is selected from: 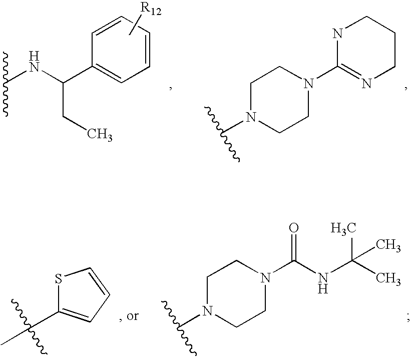
or a progdrug carbamate thereof wherein at least one of R₁ and R₂ is —COOR, wherein R₁₂ is defined as above; R is hydrogen, C₁₋₆alkyl, benzyl, or —CH(OCOCH₃)CH₃, or a pharmaceutically-acceptable salt or hydrate of said compound or prodrug carbamate; wherein the compound has an IC₅₀ for inhibiting Factor XIa of less than 3 nM.
